(19) United States
(12) Patent Application Publication
(10) Pub. No.: US 2008/0260575 A1
(43) Pub. Date: Oct. 23, 2008

(54) TWO-STAGE CATOX APPARATUS AND PROCESS

(75) Inventors: RUSSELL W. JOHNSON, ELMHURST, IL (US); PETER M. MICHALAKOS, CHICAGO, IL (US); BRIAN ELMIGER, OAK PARK, IL (US); ROBERT TOM, REDONDO BEACH, CA (US)

Correspondence Address: HONEYWELL INTERNATIONAL INC., 101 COLUMBIA ROAD, P O BOX 2245, MORRISTOWN, NJ, 07962-2245, US

(73) Assignee: HONEYWELL INTERNATIONAL INC., MORRISTOWN, NJ (US)

(21) Appl. No.: 11/736,069

(22) Filed: Apr. 17, 2007

(51) Int. Cl. A61L 9/00; B01J 19/00

(52) U.S. Cl. 422/4; 422/122

(57) ABSTRACT

Apparatus and methods for the production of a secure supply of breathable air are important under conditions where threats from chemical and biological weapons may be present. A catalytic oxidation (CATOX) system may include of at least two primary elements: (1) a catalyst to destroy toxic chemicals by oxidation, and (2) a post treatment filter (PTF, also known as a post treatment adsorbent (PTA)) to remove acidic gases that may be produced by reaction over the catalyst and also remove acidic gases that can not be oxidized. Biological materials may be destroyed by sterilization at the CATOX normal operating temperatures. The catalyst and the post treatment filter may be operated in a two-stage manner, each operating at an independent temperature.

### BACKGROUND OF THE INVENTION

The present invention generally relates to environmental control systems and, more specifically, to a two-stage system and method for providing a supply of breathable air under conditions where threats from chemical and biological weapons may be present.

In certain applications, it may be highly desirable to remove chemical and biological toxic compounds from the air. Catalysts may be used to destroy certain toxic compounds in an air stream. In order for air to be breathable, by-products of the destruction of toxic compounds must be removed. For example, nitrogen oxides (NOₓ) produced from the destruction of nitrogen containing compounds must be removed, typically through the use of a NOₓ post-treatment filter (PTF). Conventional methods heat the air that is provided to the catalyst-PTF assembly upstream of the catalytic reactor, which does not allow the operating temperature of the catalysts and PTFs to be separately optimized. As a result, large catalyst-PTF assemblies are required to adequately remove toxins from an air stream in order to produce breathable air. Such large catalyst-PTF assemblies may not be suitable in situations where there may be confined space and reduced weight requirements, such as, for example, in aircraft.

For example, U.S. Patent Publication 2003/0017090 describes an environmental control system including an isothermal catalytic oxidation (CATOX)/PTF. In this system, the air is heated and isothermally delivered to a CATOX/PTF assembly for treatment to remove chemical impurities.

As can be seen, there is a need for an apparatus and methods for the destruction of chemical and biological impurities from an air stream to yield a source of breathable air. Furthermore, there is a need for such an apparatus to occupy minimal volume and have a reduced weight as compared to conventional catalyst-PTF systems.

### SUMMARY OF THE INVENTION

In one aspect of the present invention, an apparatus for purifying air comprises a first reactor operated at a first temperature; and a second reactor downstream of the first reactor operated at a second temperature, wherein the first temperature is different from the second temperature; at least a portion of the volume in the first reactor and a portion of the volume in the second reactor contains a catalyst for the oxidation of toxicants in air; and a portion of the volume of the second reactor also includes a post treatment filter.

In another aspect of the present invention, an apparatus for removing toxicants from ambient air comprises a first heat exchanger for warming the air to a first temperature; a first reactor comprising a catalyst for oxidizing toxicants from the air warmed by the first heat exchanger to provide a first reactor effluent; a second heat exchanger for warming the first reactor effluent to a second temperature to provide a warmed first reactor effluent, the second temperature being warmer than the first temperature; a second reactor comprising a post treatment filter for removing acidic gases from the warmed second reactor effluent to provide a purified air; a third heat exchanger for cooling the purified air, the third heat exchanger being cooled with a cooling liquid provided by a vapor cycle system.

In a further aspect of the present invention, a method for producing a purified air flow from ambient air comprises destroying toxicants from the ambient air with a catalyst operated at a first temperature to provide an effluent; filtering acid gases from the effluent at a second temperature to provide the purified air; and avoiding or minimizing the generation of the acid gases by setting the first temperature lower than the second temperature.

These and other features, aspects and advantages of the present invention will become better understood with reference to the following drawings, description and claims.

### BRIEF DESCRIPTION OF THE DRAWINGS

FIG. 1 is a schematic drawing showing one embodiment of a two-stage CATOX system according to the present invention;

FIG. 2 is a schematic drawing showing an alternate embodiment of a two-stage CATOX system according to the present invention;

FIG. 3 is a schematic drawing showing an alternate embodiment of a two-stage CATOX system according to the present invention;

FIG. 4 is a schematic drawing showing an alternate embodiment of a two-stage CATOX system according to the present invention;

FIG. 5 is a graph showing the relationship between catalyst outlet temperature and reactor effluent; and

FIG. 6 is a flow chart describing a method according to one embodiment of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides apparatus and methods for ensuring a secure supply of breathable air under conditions where threats from chemical and biological weapons may be present. The catalytic oxidation (CATOX) system may be comprised of at least two primary elements: (1) a catalyst to destroy toxic chemicals by oxidation, and (2) a post treatment filter (PTF, also known as a post treatment adsorbent (PTA)) to remove acidic gases that may be produced by reaction over the catalyst and also remove acidic gases that can not be oxidized. Biological materials present in the air may be destroyed by sterilization at the CATOX normal operating temperatures. These two elements may be operated in a two-stage manner, each operating at an independent temperature.

The CATOX system of the present invention may be useful in any enclosed environment where a secure supply of breathable air is required. For example, the CATOX system of the present invention may be useful in vehicles, such as tanks, airplanes, helicopters, trains, ships and the like, and buildings, such as office buildings, factories, shelters and the like.

As will be discussed in more detail below, a PTF may exhibit better performance if operated at a relatively higher temperature as compared to the temperature at which catalytic oxidation may take place. However, excessive nitrogen oxides may be produced if the catalytic conversion occurs at this relatively higher temperature. Therefore, to achieve adequate oxidation of the chemical toxicants and adequate removal of nitrogen oxides and other byproducts of the chemical oxidation (such as, for example, HCl and SO₂), a large combined catalyst-PTF assembly is required. Using a two-stage CATOX process, according to the present invention, the size and weight of the CATOX assembly may be significantly reduced, as compared to conventional CATOX assemblies, while not reducing the effectiveness of the CATOX process.

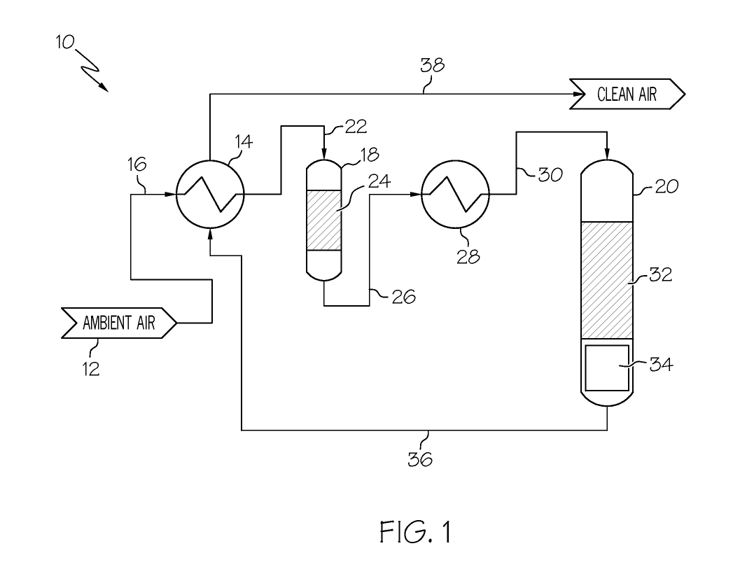

Referring to FIG. 1, there is shown one example of a CATOX assembly 10 according to the present invention. Ambient air 12 may be supplied to a lower temperature heat exchanger 14 via a line 16. The lower temperature heat exchanger 14 may be used to heat the incoming ambient air 12 with energy that may include, for example, one or more of the following sources: recuperation from catalytic reactors 18, 20 (as discussed in more detail below), electrical, fuel combustion or exhaust heat.

Heated air may pass through line 22 into a lower temperature catalytic reactor 18 which may be filled or partially filled with a catalytic composition 24 capable of oxidizing or decomposing toxic compounds, including nitrogen-containing compounds. The catalytic composition 24 may be any of the various known catalytic compositions that may be used to oxidize toxic compounds, such as those disclosed in U.S. Pat. Nos. 5,292,704, 5,720,931, 6,503,462 and 7,132,086, each of which are hereby incorporated by reference.

Most toxicant materials may be destroyed in the lower temperature catalytic reactor 18. For example, greater than 50%, and often greater than 90% of toxicant materials may de destroyed in the lower temperature catalytic reactor 18.

The lower temperature catalytic reactor 18 may operate at a sufficiently lower temperature to generate a very low yield of nitric oxide and nitrogen dioxide from nitrogen-containing organic material and cyano-inorganic materials. For example, the lower temperature catalytic reactor 18 may operate at a temperature between about 100° C. and about 300° C., often between about 200° C. and about 280° C.

The effluent from the lower temperature catalytic reactor 18 may be conducted through a line 26 to a higher temperature heat exchanger 28. As discussed below, the higher temperature heat exchanger 28 may raise the temperature of the effluent in line 26 to a temperature higher than the temperature generated by the lower temperature heat exchanger 14. Similar to the lower temperature heat exchanger 14, the higher temperature heat exchanger 28 may be a used to heat the effluent in line 26 with energy that may include, for example, one or more of the following sources: recuperation from catalytic reactors 18, 20, electrical, fuel combustion or exhaust heat.

The warmed effluent from the higher temperature heat exchanger 28 may pass through a line 30 to a higher temperature catalytic reactor 20 which may be a vessel filled or partially filled with a catalytic composition 32. The catalytic composition 32 in the higher temperature heat exchanger 20 may be the same or different from the catalytic composition 24 in the lower temperature catalytic reactor 18. In both cases, the catalytic compositions 24, 32 may be capable of destroying toxic chemicals by oxidation.

The temperature of the warmed effluent in line 30 being delivered to the higher temperature catalytic reactor 20 may be higher than the temperature of the heated air in line 22 being delivered to the lower temperature catalytic reactor 18. For example, the higher temperature catalytic reactor 20 may operate at a temperature from about 200° C. to about 500° C., often between about 280° C. and about 320° C. The higher temperature catalytic reactor 20 may have a sufficiently higher temperature such that at least about 99.9999% of toxic compounds may be destroyed by this combination of catalytic reactors.

The higher temperature catalytic reactor 20 may also contain a post treatment filter (PTF) 34 capable of removing acidic gases that may include nitric oxide and nitrogen dioxide. The formulation for the PTF 34 may be similar to the formulation disclosed in U.S. Pat. No. 7,132,086, incorporated herein by reference. However, the formulation is not so limited. Other formulations may be used, such as those disclosed by Shimada et al. in European Patent Application No. 0 625 368 A1, for example. Additionally, a platinum impregnated post treatment adsorbent (not shown) may be used in the higher temperature reactor 20. The post treatment adsorbent may have both catalytic activity for destroying toxicants and adsorption capacity for removing acidic gases.

The effluent from the PTF 34 may pass through a line 36 to the lower temperature heat exchanger 14, where heat may be recuperated before discharging the clean air through line 38. Unlike single reactor stage systems, which may operate a catalytic reactor at a single temperature or in an adiabatic manner, the CATOX assembly 10 may destroy toxicants at a first, lower temperature (e.g., the temperature of the heated air in line 22) with minimal generation of acid gases. Then, at a second, higher temperature (e.g., the temperature of the effluent in line 30), the CATOX assembly 10 may destroy the remaining toxicants. The PTF downstream of the second catalyst may then remove a smaller quantity of acid gases and therefore reduce system size.

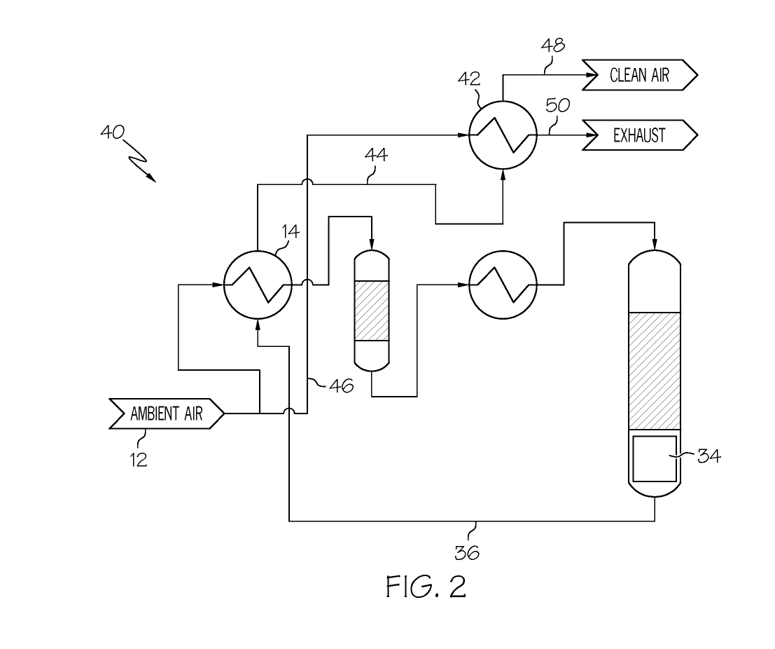

Referring now to FIG. 2, there is shown a CATOX assembly 40 according to an alternate embodiment of the present invention. The CATOX assembly 40 may use the same equipment as the CATOX assembly 10 of FIG. 1 with the addition of an air cooling heat exchanger 42.

After treatment with the PTF 34 (as described above with reference to FIG. 1), the treated air may pass through line 36 to the lower temperature heat exchanger 14, where heat may be recuperated before discharging the clean air through line 44 to the air cooling heat exchanger 42. The air cooling heat exchanger 42 may be an air/air heat exchanger using ambient air 12 delivered via line 46 as the cooling source. Clean cooled air may be provided through line 48 and the heated ambient air from line 46 may be discharged as exhaust via line 50.

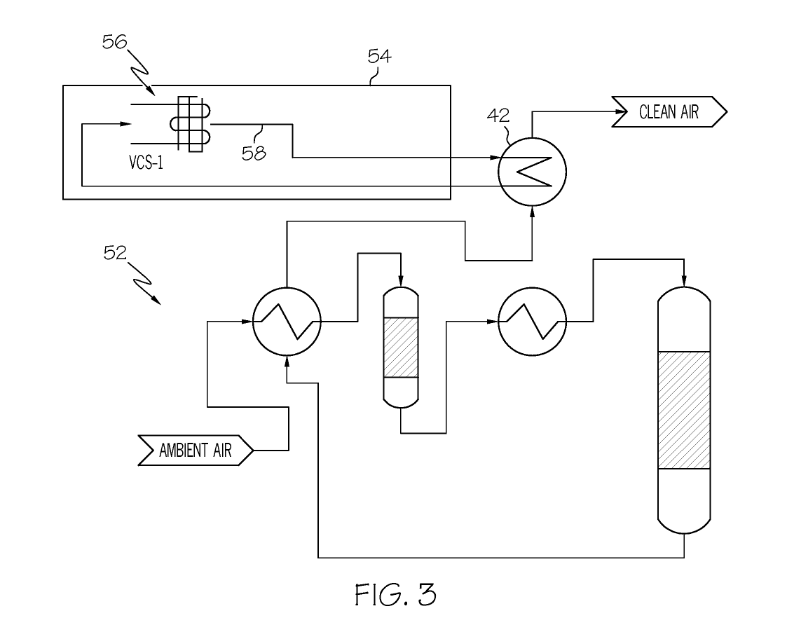

Referring now to FIG. 3, there is shown a CATOX assembly 52 according to an alternate embodiment of the present invention. The CATOX assembly 52 may use the same equipment as the CATOX assembly 40 of FIG. 2 with the addition of a vapor cycle cooled liquid cooling loop 54. In the above embodiment of FIG. 2, ambient air may be used as the coolant in the cooling air heat exchanger 42. However, in ground operations, where the ambient air may be too warm, an alternate source of coolant may be needed for the cooling air heat exchanger 42. In these cases, a vapor cycle system 56 may be used to cool a liquid (not shown) that may be delivered to the cooling air heat exchanger 42 via line 58. When a cooled liquid is fed to the cooling air heat exchanger 42, a liquid/air heat exchanger may be used as the cooling air heat exchanger 42.

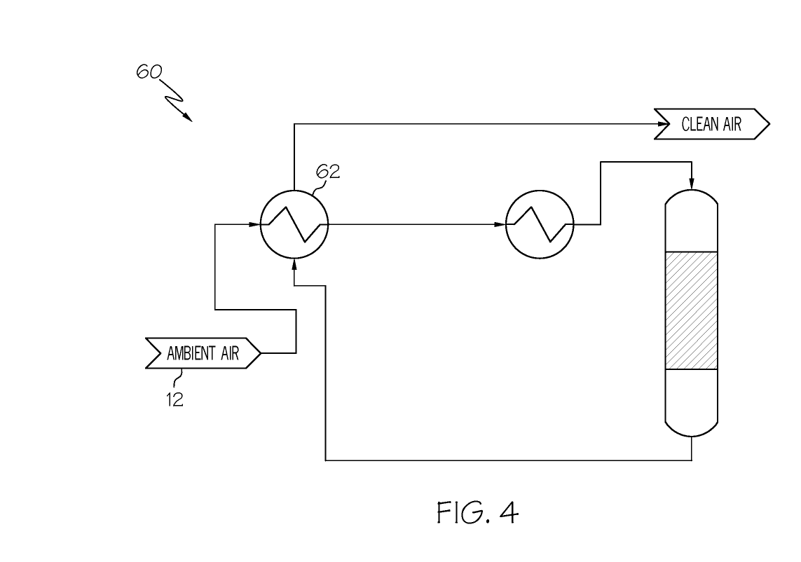

Referring to FIG. 4, there is shown a CATOX assembly 60 according to an alternate embodiment of the present invention. The CATOX assembly 60 may use the same equipment as the CATOX assembly 10 of FIG. 1, except that the lower temperature heat exchanger 14 (see FIG. 1) and the lower temperature catalytic reactor 18 (see FIG. 1) may be combined into a combined lower temperature heat exchanger/reactor 62. As ambient air 12 flows through the combined lower temperature heat exchanger/reactor 62, the ambient air 12 may contact a catalyst (not shown, however similar to catalyst 24 of FIG. 1) coated onto the flow path of the ambient air 12 through the combined lower temperature heat exchanger/reactor 62.

While the above FIGS. 1 through 4 describe embodiments of the present invention, such embodiments should only be seen as exemplary embodiments and are not meant to limit the scope of the present invention. Broadly, the present invention may be drawn to a two stage CATOX apparatus and process, wherein the at least one catalytic reaction may be at a temperature lower than the post treatment filtration. Therefore, modifications of FIGS. 1 through 4 may be contemplated as being within the scope of the present invention.

For example, the embodiment of FIG. 1 may be modified such that all of the catalyst 24 may be in the lower temperature catalytic reactor 18 and all of the PTF 34 may be in the higher temperature catalytic reactor 32 (which, in this case, the higher temperature catalytic reactor 32 would not be a catalytic reactor at all, but instead, a vessel for containing the PTF 34). In this case, the higher temperature heat exchanger 28 may produce an effluent to the PTF 34 via line 30 at a temperature of over 350° C., thereby removing the acid gas produced by the lower temperature catalytic reactor 32, which, in this case, may be operated at a temperature of about 275° C.

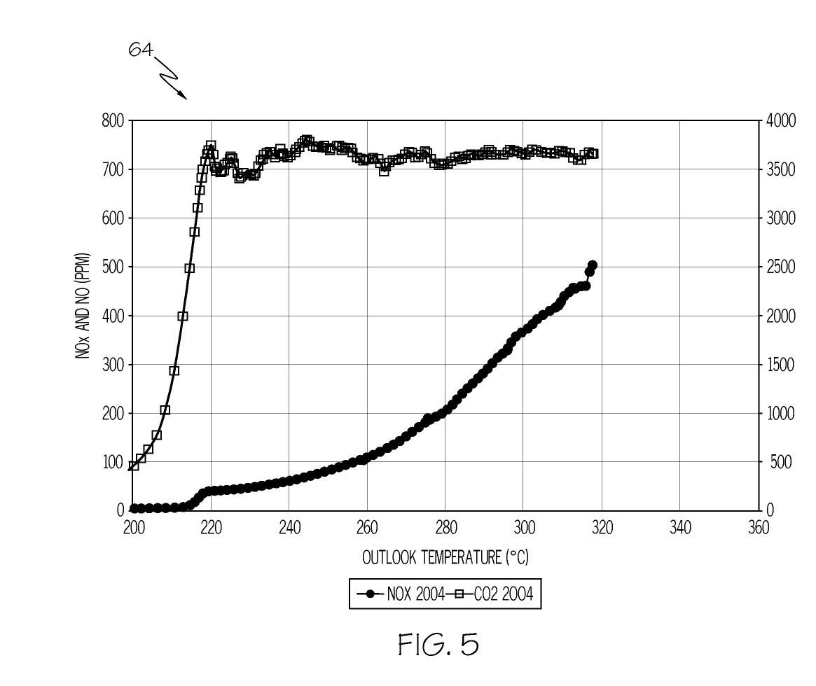

Referring now to FIG. 5, there is shown a graph 64 describing the relationship between catalyst outlet temperature and reactor effluent. The data shown in this graph 64 was obtained from an experiment wherein an admixture of 1,800 parts per million by volume (ppm) of acetonitrile (CH₃CN) and air was charged to a catalytic reactor as the temperature was changed. The CO₂ reactor effluent (ppm) and the NOₓ and NO effluent (ppm) were measured as a function of outlet temperature (° C.).

The carbon dioxide generated shows that the point of high conversion was obtained at a reactor temperature above about 220° C. In other words, increasing the temperature beyond about 220° C. did not increase the carbon dioxide effluent. Therefore, it can be assumed that a high level of conversion but not necessarily >99.9999% of acetonitrile to carbon dioxide was obtained at a temperature of at least about 220° C. At this temperature (220° C.), only about 40 ppm of NOₓ was produced. The remaining nitrogen in the acetonitrile was converted to less toxic materials, such as nitrogen gas (N₂) and nitrous oxide (N₂O). It is much more desirable to have these materials, rather than NOₓ, present in the breathable air.

Several toxic compounds that the CATOX system is required to treat may require a higher temperature for catalytic oxidation. For this reason, the catalyst must be operated at a higher temperature. However, based on laboratory data of FIG. 5, at about 300° C. (a temperature suitable for the conversion of a broad spectrum of toxicants), approximately 370 ppm of NOx was produced. By utilizing the two-stage apparatus and methods of the present invention, a first catalytic reaction may be conducted at 220° C., producing about 40 ppm NOx. As discussed above, this catalytic reaction destroyed all of the acetonitrile present in the air stream. A second catalytic reaction may be conducted at 300° C. to remove other compounds that do not contain nitrogen and are less reactive at lower temperatures. In this second catalytic reaction, little or no NOx may be generated because the acetonitrile was destroyed in the first step at 220° C. The result may be effective catalytic oxidation at 300° C. without the associated NOx production (which would be about 370 ppm, a 9-fold increase from the two stage process production of 40 ppm).

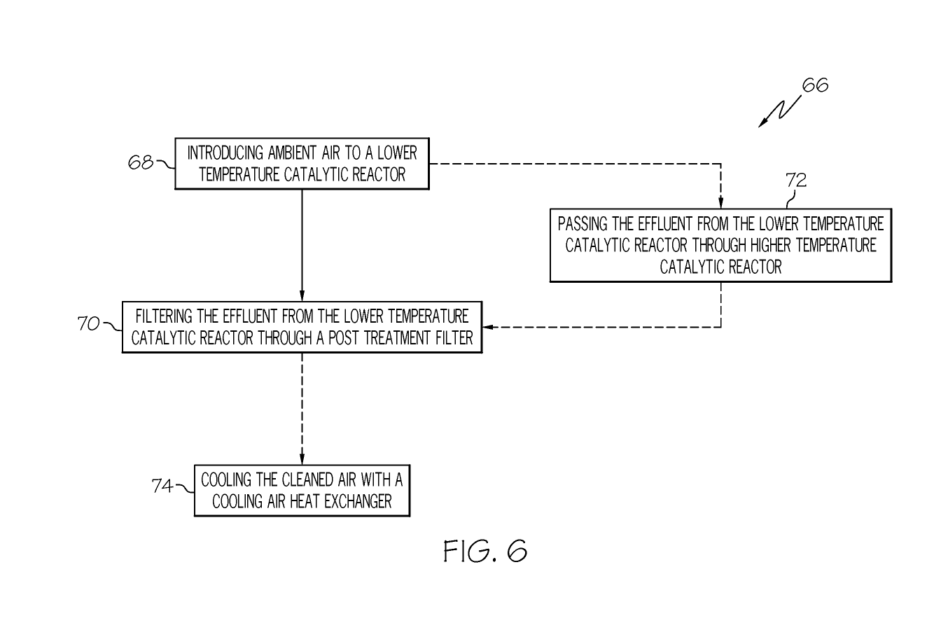

Referring to FIG. 6, there is shown a flow chart describing a method 66 for producing a purified air flow according to one embodiment of the present invention. Broadly, the method 66 may include a first step 68 of introducing ambient air to a lower temperature catalytic reactor (e.g., lower temperature catalytic reactor 18) at a first temperature and a second step 70 of filtering the effluent from step 68 through a post treatment filter (e.g., post treatment filter 34) at a second temperature, wherein the first temperature is lower than the second temperature. The method 66 may include, as an optional step, a step 72 of passing the effluent from step 68 through a higher temperature catalytic reactor (e.g., higher temperature catalytic reactor 20) prior to the PTF step 70. Furthermore, the method 66 may include a further optional step 74 of cooling the cleaned air with a cooling air heat exchanger (e.g., cooling air heat exchanger 42). The cooling air heat exchanger may have, as a cooling source, ambient air or a liquid cooled by, for example, a vapor cycle system (e.g., vapor cycle system 56).

It should be understood, of course, that the foregoing relates to exemplary embodiments of the invention and that modifications may be made without departing from the spirit and scope of the invention as set forth in the following claims.

We claim:

1. An apparatus for purifying air comprising:
a first reactor operated at a first temperature; and
a second reactor downstream of the first reactor operated at a second temperature, wherein
the first temperature is different from the second temperature;
at least a portion of the volume in the first reactor contains a catalyst for the oxidation or decomposition of toxicants in air; and
at least portion of the second reactor includes a post treatment filter.

2. The apparatus according to claim 1, wherein the first temperature is lower than the second temperature.

3. The apparatus according to claim 1, further comprising a first heat exchanger for warming the air to the first temperature.

4. The apparatus according to claim 3, wherein the first heat exchanger and the first reactor are combined into a combined heat exchanger/reactor.

5. The apparatus according to claim 1, further comprising a second heat exchanger for warming the air to the second temperature.

6. The apparatus according to claim 1, wherein at least a portion of the volume in the first reactor and at least a portion of the volume in the second reactor both contain the same or different catalyst.

7. The apparatus according to claim 1, wherein purified air from the second reactor is passed through the first heat exchanger to recuperate heat therefrom.

8. The apparatus according to claim 1, further comprising a third heat exchanger for cooling purified air from the second reactor.

9. The apparatus according to claim 8, wherein the third heat exchanger uses ambient air as a cooling source.

10. The apparatus according to claim 8, further comprising a vapor cycle system, the vapor cycle system providing a cooled liquid as a cooling source for the third heat exchanger.

11. The apparatus according to claim 1, wherein the post treatment filter removes acidic gases from the air.

12. The apparatus according to claim 1, wherein the second reactor includes a platinum impregnated post treatment adsorbent, the post treatment adsorbent having both catalytic activity and adsorption capacity.

12. The apparatus according to claim 1, wherein the first temperature is between about 100° C. to about 300° C. and the second temperature is between about 200° C. to about 500° C.

13. An apparatus for removing toxicants from ambient air comprising:
a first heat exchanger for warming the air to a first temperature;
a first reactor comprising a catalyst for oxidizing toxicants from the air warmed by the first heat exchanger to provide a first reactor effluent;
a second heat exchanger for warming the first reactor effluent to a second temperature to provide a warmed first reactor effluent, the second temperature being warmer than the first temperature;
a second reactor comprising a post treatment filter for removing acidic gases from the warmed second reactor effluent to provide a purified air;
a third heat exchanger for cooling the purified air, the third heat exchanger being cooled with a cooling liquid provided by a vapor cycle system.

14. The apparatus according to claim 13, wherein the first reactor provides at least 50% destruction of toxicant materials.

15. The apparatus according to claim 13, further comprising a second catalyst in the second reactor.

16. A method for producing a purified air flow from ambient air, the method comprising:
destroying toxicants from the ambient air with a catalyst operated at a first temperature to provide an effluent;
filtering acid gases from the effluent at a second temperature to provide the purified air; and
minimizing the generation of the acid gases by setting the first temperature lower than the second temperature.

17. The method according to claim 16, further comprising destroying toxicants from the effluent with a second catalyst operated at a temperature greater than the first temperature.

18. The method according to claim 16, further comprising cooling the purified air with a cooling air heat exchanger.

19. The method according to claim 1, further comprising:
warming the ambient air to the first temperature with a first heat exchanger; and
warming the effluent to the second temperature with a second heat exchanger.

20. The method according to claim 1, wherein the method provides a conversion of about 99.9999% of the nitrogen-containing organic materials and the cyano-inorganic materials in the ambient air.
